(19) United States
(12) Patent Application Publication
(10) Pub. No.: US 2008/0208122 A1
(43) Pub. Date: Aug. 28, 2008

(54) Controlling the Retraction of a Needle in an Auto Retractable Syringe

(75) Inventors: Graeme Francis Walton, New South Wales (AU); Allan Walsh, New South Wales (AU)

Correspondence Address: BROOKS KUSHMAN P.C., 1000 TOWN CENTER, TWENTY-SECOND FLOOR, SOUTHFIELD, MI, 48075, US

(73) Assignee: GLOBAL MEDISAFE HOLDINGS LIMITED, The Hill, NSW (AU)

(21) Appl. No.: 11/910,924

(22) PCT Filed: Apr. 8, 2006
(86) PCT No.: PCT/AU2006/000455

§ 371 Date: Oct. 8, 2007

(30) Foreign Application Priority Data
Apr. 8, 2005 (AU) 2005901745

(51) Int. Cl. A61M 5/178; A61M 5/32

(52) U.S. Cl. 604/118; 604/195

(57) ABSTRACT

Means to control the retraction of an auto-retractable safety syringe to minimise splashback or splatter is disclosed. The end of a hollow plunger (11) is covered by a sealing member (14) that, upon retraction, is dislodged so that the needle assembly and seal enters the hollow plunger. Three methods by which the auto-retraction is controlled are disclosed. Firstly there is provided a taper within the inside of the hollow plunger which creates varying resistance of movement to the sealing member. Secondly there is provided a bleed hole (15) at the upper end of the plunger, whereby the air is allowed to escape. Thirdly, there is provided on or more grooves (16) in the periphery of the plunger seal so that air is allowed to be released, thereby reducing the pressure in the plunger and hence the possibility of splashback.

### TECHNICAL FIELD

This invention relates to a safety syringe having an auto retractable needle for use in the medical or dental profession or in personal drug administration so that the physician, surgeon or other needle operator might be protected from injury by the needle after its use. In particular, it relates to means for controlling the rate at which auto retraction occurs to minimise so-called splash back.

### BACKGROUND

The danger of injury and possible infection from the HIV or hepatitis B virus to medical practitioners using needles in the normal course of their business is well documented.

Thus, there is a need to protect medical staff from needle stick injuries. In a time when it is known that there are many blood born diseases, it is becoming more critical to protect the medical profession against such diseases.

Patients also deserve the same protection against such diseases. If a medical practitioner receives a needle stick injury prior to giving an injection, any blood disease that medical practitioner has can be passed on to the patient.

Furthermore, persons who are in the habit of administering drugs to themselves run a severe risk of contacting either of the specified viruses, or indeed contacting other viruses if a needle, once used, is reused in an unsterilized form.

There have been many proposals aimed at reducing the number of so-called needle-stick injuries and various attempts have been made to provide a safe system for disposal of such needles once used, but such prior proposals have had deficiencies.

While it is known there are auto retractable syringes on the market, these syringes often suffer from the disadvantage that splash back or splatter can occur, when the needle is caused to be retracted.

Thus without adequate control of the rate of retraction, there can be undesirable splash back from fluid retained in the needle. Whilst at first it might be thought useful to simply reduce the strength of the spring, which is the usual means of causing the needle to be retracted, in practice this is not feasible since the spring has to be sufficient to move the needle and associated hub back into plunger along with the plunger seal, which needs to be present.

Another major problem with auto retractable syringes is that in order to activate the auto retraction it is generally required that the plunger of the syringe be pushed harder beyond the pressure at which the injection would normally be required. Therefore, if the needle of the syringe is drawn from the patient before the auto retraction is activated then it matters little how violent the initial kick-back is. On the other hand, if the auto retraction of the needle in the syringe is activated whilst the needle is in the patient, either accidentally or deliberately, it can be very uncomfortable or cause damage to the tissue in which, the needle is inserted. Accordingly, it would be preferable if there were an improved method associated with auto retractable needles whereby the initial movement of the needle, or kickback, was indeed dampened.

### OBJECT OF THE INVENTION

It is therefore an object of the present invention to provide means whereby the rate of travel of the needle in an auto retractable syringe is controlled to the extent at least that splashback or splatter is minimized. It is a further object of the invention to ameliorate the forgoing disadvantages found in auto retractable syringes where the needle is uncontrollably retracted, causing discomfort or injury if performed whilst the needle is still in the patient.

At the very least, the invention provides alternate means for providing an auto retractable syringe to protect against accidental injury arising from unwanted contact with the exposed needle prior to use and once the syringe has been used.

Although the following description generally refers to a syringe of conventional size, no such limitation is intended thereby, and reference to a syringe is meant to encompass any other needle/syringe combination or needle alone including slimline syringes, where, by suitable adaptation, the invention may also be usefully applied.

### DISCLOSURE OF THE INVENTION

The invention in one broad form provides means for controlling the rate at which an auto retractable needle is caused to retract in order to minimise splash back or splatter.

Examples of syringes with auto retractable needles to which the invention is applicable include those described in our co-pending International patent applications PCT/AU2005/000510, PCT/AU2005/001132 and PCTAU/2006/000331 each of which is wholly incorporated herein by way of reference. However, it should not be construed that the invention is thereby limited to only these syringes, but includes all syringes where, by suitable modification, the invention may also be usefully included.

In order to slow the retraction, at least initially, to a rate at which splatter is less likely to occur three methods have been found useful all of which rely on the principle of controlling the airflow in the plunger as the needle etc is retracted. Thus when the needle hub is dislodged at the time of auto retraction, the spring pressure pushes the needle hub up pushing the already dislodged inner seal inside the plunger.

One method of controlling the airflow is to provide a taper within the inside of the hollow plunger, so that the needle hub is caused to start its journey more slowly by virtue of the initial greater resistance.

A second method is to provide a bleed hole at the upper end of the plunger, whereby the air can be allowed to escape, thus reducing the likelihood of pressure build up in the plunger which would otherwise only be released through the needle itself, thus causing splatter.

A third method is to provide one or more grooves in the periphery of the plunger seal so that air is allowed to be released, thereby reducing the pressure in the plunger and hence the possibility of splashback.

Thus the inner seal sealing the plunger in a preferred embodiment has air grooves cut through to allow a slow return of the needle inside the plunger eliminating blood splash, at the time of auto needle retraction after injection has taken place.

A more sophisticated solution utilises a tapered hole in the plunger. When the inner seal is retracted by syringe pressure after injection, the tapered bore allows ever increasing air to escape around the inner seal. This allows the needle retraction to start slowly but will cause the needle to increase in velocity.

One example of an auto-retractable syringe wherein the invention may be applied is in auto retractable safety syringe having a syringe barrel which has a hollow plunger located therein, the plunger having piston means associated therewith so as to be sealingly slideable within the barrel, the syringe further having a needle cartridge assembly located in the barrel, the needle cartridge assembly comprising a cartridge housing a needle having a needle hub, the needle cartridge assembly further having auto retractable means whereby the needle may be ejected from the needle cartridge assembly and fully retracted into the hollow plunger upon activation of the auto retractable means, the needle cartridge assembly being initially releasably engaged on the end of the plunger in a retracted state within the barrel prior to use, and movable to and engageable with the end of the barrel where the needle extends therefrom in an in-use position for performing an injection, and during which time the plunger is withdrawn from engagement with the needle cartridge assembly, so as to function in known manner as a plunger for taking fluid into the barrel and causing it to be injected into a patient, and wherein subsequent to completion of the injection the plunger is depressed further to cause the needle cartridge assembly to eject the needle from the needle cartridge assembly by activating the auto retractable means such that the needle is caused to retract into the hollow plunger.

Preferably the hollow plunger of an auto retractable safety syringe of this type has is initially sealed by a disc in the form of a seal across the end of the plunger, which seal is broken or ruptured during auto retraction. Preferably the seal is made of a flexible rubber or rubber like material.

Alternatively, a safety syringe to which the invention may be applied is in the form of a safety syringe incorporating a removable auto retractable exchange needle assembly therein, the safety syringe having a syringe barrel which has a hollow plunger slideably located therein, the hollow plunger having a proximal and distal end with piston means associated therewith at its distal end so as to be sealingly slideable within the barrel, the needle assembly having means for removable attachment thereof to the end of the barrel, the needle assembly comprising a gland nut, needle and needle cap to protect the needle prior to use, with a recess in the gland nut to hold a needle hub of the needle and a spring disposed over said needle hub and acting between the needle hub and the gland nut, the spring remaining under compression during use, the needle hub having sealing means associated therewith to prevent leakage between the needle hub and the gland nut, a housing disposed on one side of said gland nut to accommodate a dowel pin, said needle hub having a groove around its circumference in which an end of said dowel pin mates so as to retain the needle hub in the gland nut during use against the compressive action of the spring, and wherein said distal end of the hollow plunger has a sealing stopper, the plunger contacting in a first stage said needle assembly upon completion of an injection and when further depressed activating auto retraction of the needle hub by acting upon the needle assembly so as to cause said end of dowel pin to be dislodged from said groove to release the needle hub by reaching a second stage, said sealing stopper dislodging from said distal end of the plunger when it reaches the second stage thereby releasing said needle hub and hence allowing the needle and spring to retract inside said hollow plunger under the influence of the spring.

Alternatively, a further embodiment of an auto-retractable safety syringe to which the invention may be applied is in the form of an auto-retractable safety syringe incorporating an auto retractable needle assembly therein, the safety syringe having a syringe barrel which has a hollow plunger slideably located therein, the hollow plunger having a proximal and distal end with piston means associated therewith at its distal end so as to be sealingly slideable within the barrel, the needle assembly comprising a needle and a needle hub, with a recess in the end of the barrel to hold the needle hub of the needle and a spring disposed over said needle hub and acting between the needle hub and the end of the barrel, the spring remaining under compression during use, the needle hub having sealing means associated therewith to prevent leakage between the needle hub and the end of the barrel, said needle hub having a groove or recess around its circumference in which a locking means mates so as to retain the needle hub in the end of the syringe barrel during use against the compressive action of the spring, and wherein said distal end of the hollow plunger has a sealing stopper, the plunger contacting in a first stage said needle assembly upon completion of an injection and when further depressed activating auto retraction of the needle hub by acting upon the locking means so as to cause said locking means to be dislodged from said groove or recess to release the needle hub by reaching a second stage, said sealing stopper dislodging from said distal end of the plunger when it reaches the second stage thereby releasing said needle hub and hence allowing the needle and spring to retract inside said hollow plunger under the influence of the spring.

Preferably, the needle and needle hub are housed in a needle gland, which may be interchangeably connected to the barrel of the syringe. More preferably, the means by which the needle assembly is removably attached to the barrel is provided by having the gland bush fitted to the end of the barrel by threaded means. This includes a standard screw fitting or a so-called luer-lock fitting.

### BRIEF DESCRIPTION OF THE DRAWINGS

The invention may be better understood from the following non-limiting description of preferred embodiments, in which:

FIG. 1 is a cross sectional side view of a plunger for use in an auto retractable syringe, in which the rate of travel of the retracted needle is controlled by controlling the air flow by a taper in the plunger;

FIG. 2 is a cross sectional side view of a plunger for use in an auto retractable syringe, in which the rate of travel of the retracted needle is controlled by controlling the air flow by a grooves in the plunger seal; and

FIG. 3 is a cross sectional side view of a plunger for use in an auto retractable syringe, in which the rate of travel of the retracted needle is controlled by controlling the air flow by means of a bleed hole in the plunger;

### DETAILED DESCRIPTION OF THE DRAWINGS

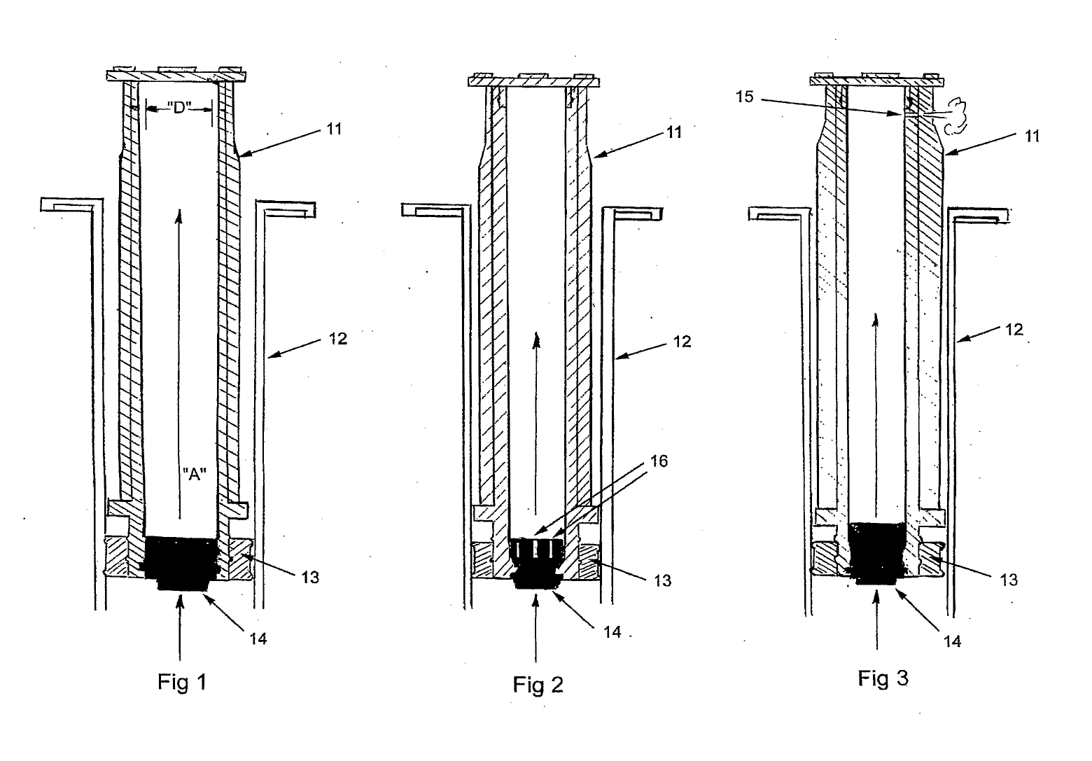

Referring generally to each of FIGS. 1 to 3, there is shown a plunger 11 for an auto retractable syringe and needle combination (not fully shown).

The plunger 11 is hollow and slides within the barrel 12 of the syringe. A piston member 13 is located between the plunger 11 and barrel 12. Each plunger 11 is provided with a sealing member 14, which allows the plunger to function normally in use, prior to auto retraction taking place.

Referring specifically to FIG. 1, there is shown a taper to the walls of the plunger 11, which because it tapers away from the seal 14, shown by the wider dimension “D” near the upper end of the plunger 11, allows air to move more slowly initially, because of higher initial resistance, as the seal 14 is caused to move inwardly (along line “A”, during auto retraction and then to increase as the resistance to the moving seal 14 from the widening walls is less as the seal 11 travels up the plunger 11. This means that there is less likelihood of splatter or splash back occurring initially whilst the end of the needle (not shown) remains in the in-use position, ie outside the barrel 12 of the syringe.

Referring to FIG. 2, there is shown in detail a portion of the end of a plunger 11. Similar features utilise the same reference numerals as in FIG. 1. In this case, the inner seal 14 has air grooves 16 cut in the periphery thereof to allow the egress of air during auto retraction to control the effect thereof as described above.

Turning to FIG. 3, where again similar features to those found in FIGS. 1 and 2 utilise the same reference numerals, the plunger 11 in this case is provided with a bleed hole 15 located away from the region of the inner seal 14. Again this feature allows the air to pass from the inner regions of the plunger 11 during the initial stages of auto retraction and in particular in this case clearly provides for air to leave by that means rather than cause pressure build up which would otherwise only be released by travelling back through the hollow needle (not shown). In this way, the pressure does not need to be relieved via the needle, which would cause spatter.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

1. An auto retractable safety syringe having an auto-retractable needle located therein, wherein the rate at which an auto retractable needle is caused to retract is controlled so as to minimize splash back or splatter, the syringe being of the type wherein the end of a hollow plunger is initially covered by a sealing member, and where upon effecting auto retraction of a needle or needle assembly containing a needle, the seal is caused to be dislodged so that the needle or needle assembly together with the seal enters the hollow plunger, wherein in order to slow the retraction of the needle or a needle assembly containing said needle, at least initially, to a rate at which splatter is less likely to occur, control of the rate of auto retraction of the needle is achieved by controlling the airflow in the plunger as the needle, or needle assembly, is retracted.

2. A method of controlling the airflow in an auto retractable safety syringe of the type according to claim 1, which is provided by a taper within the inside of the hollow plunger, so that the needle hub is caused to start its journey more slowly by virtue of the initial greater resistance provided by the initially narrower diameter of the hollow plunger near the region of the seal.

3. A method of controlling the airflow in an auto retractable safety syringe of the type according to claim 1, in which there is provided a bleed hole at the upper end of the plunger, whereby the air is allowed to escape, thus reducing the likelihood of pressure build up in the plunger which would otherwise only be released through the needle itself, thus causing splatter.

4. A method of controlling the airflow in an auto retractable safety syringe of the type according to claim 1, wherein there is provided one or more grooves in the periphery of the plunger seal so that air is allowed to be released, thereby reducing the pressure in the plunger and hence the possibility of splash back.

5. A method of controlling the airflow in an auto-retractable safety syringe according to claim 4, wherein the inner seal sealing the plunger has air grooves cut through to allow a slow return of the needle inside the plunger eliminating blood splash at the time of auto needle retraction after injection has taken place.

6. An auto-retractable syringe having a syringe barrel which has a hollow plunger located therein, the plunger having piston means associated therewith so as to be sealingly slideable within the barrel, the syringe further having a needle cartridge assembly located in the barrel, the needle cartridge assembly comprising a cartridge housing a needle having a needle hub, the needle cartridge assembly further having auto retractable means whereby the needle may be ejected from the needle cartridge assembly and fully retracted into the hollow plunger upon activation of the auto retractable means, the needle cartridge assembly being initially releasably engaged on the end of the plunger in a retracted state within the barrel prior to use, and movable to and engageable with the end of the barrel where the needle extends therefrom in an in-use position for performing an injection, and during which time the plunger is withdrawn from engagement with the needle cartridge assembly, so as to function in known manner as a plunger for taking fluid into the barrel and causing it to be injected into a patient, and wherein subsequent to completion of the injection the plunger is depressed further to cause the needle cartridge assembly to eject the needle from the needle cartridge assembly by activating the auto retractable means such that the needle is caused to retract into the hollow plunger, and wherein the hollow plunger is initially sealed by a disc in the form of a seal across the end of the plunger, which seal is broken or ruptured during auto retraction, and wherein the rate at which an auto retractable needle is caused to retract is controlled so as to minimize splash back or splatter, wherein control of the rate of auto retraction of the needle is achieved by controlling the airflow in the plunger as the needle, or needle assembly, is retracted.

7. An auto-retractable syringe wherein the method of claim 2 is utilized to control the rate of auto retraction of the needle or needle assembly.

8. An auto retractable safety syringe incorporating a removable auto retractable exchange needle assembly therein, the safety syringe having a syringe barrel which has a hollow plunger slideably located therein, the hollow plunger having a proximal and distal end with piston means associated therewith at its distal end so as to be sealingly slideable within the barrel, the needle assembly having means for removable attachment thereof to the end of the barrel, the needle assembly comprising a gland nut, needle and needle cap to protect the needle prior to use, with a recess in the gland nut to hold a needle hub of the needle and a spring disposed over said needle hub and acting between the needle hub and the gland nut, the spring remaining under compression during use, the needle hub having sealing means associated therewith to prevent leakage between the needle hub and the gland nut, a housing disposed on one side of said gland nut to accommodate a dowel pin, said needle hub having a groove around its circumference in which an end of said dowel pin mates so as to retain the needle hub in the gland nut during use against the compressive action of the spring, and wherein said distal end of the hollow plunger has a sealing stopper, the plunger contacting in a first stage said needle assembly upon completion of an injection and when further depressed activating auto retraction of the needle hub by acting upon the needle assembly so as to cause said end of dowel pin to be dislodged from said groove to release the needle hub by reaching a second stage, said sealing stopper dislodging from said distal end of the plunger when it reaches the second stage thereby releasing said needle hub and hence allowing the needle and spring to retract inside said hollow plunger under the influence of the spring, and wherein the hollow plunger is initially sealed by a disc in the form of a seal across the end of the plunger, which seal is broken or ruptured during auto retraction, and wherein the rate at which an auto retractable needle is caused to retract is controlled so as to minimize splash back or splatter.

9. An auto-retractable syringe according to claim 8 wherein control of the rate of auto retraction of the needle is achieved by controlling the airflow in the plunger as the needle, or needle assembly, is retracted.

10. An auto-retractable syringe wherein the method of claim 3 is utilized to control the rate of auto retraction of the needle or needle assembly.

11. An auto-retractable safety syringe incorporating an auto retractable needle assembly therein, the safety syringe having a syringe barrel which has a hollow plunger slideably located therein, the hollow plunger having a proximal and distal end with piston means associated therewith at its distal end so as to be sealingly slideable within the barrel, the needle assembly comprising a needle and a needle hub, with a recess in the end of the barrel to hold the needle hub of the needle and a spring disposed over said needle hub and acting between the needle hub and the end of the barrel, the spring remaining under compression during use, the needle hub having sealing means associated therewith to prevent leakage between the needle hub and the end of the barrel, said needle hub having a groove or recess around its circumference in which a locking means mates so as to retain the needle hub in the end of the syringe barrel during use against the compressive action of the spring, and wherein said distal end of the hollow plunger has a sealing stopper, the plunger contacting in a first stage said needle assembly upon completion of an injection and when further depressed activating auto retraction of the needle hub by acting upon the locking means so as to cause said locking means to be dislodged from said groove or recess to release the needle hub by reaching a second stage, said sealing stopper dislodging from said distal end of the plunger when it reaches the second stage thereby releasing said needle hub and hence allowing the needle and spring to retract inside said hollow plunger under the influence of the spring, and wherein the needle and needle hub are housed in a needle gland, which may be interchangeably connected to the barrel of the syringe, and wherein the hollow plunger is initially sealed by a disc in the form of a seal across the end of the plunger, which seal is broken or ruptured during auto retraction, and wherein the rate at which an auto retractable needle is caused to retract is controlled so as to minimise splash back or splatter.

12. An auto-retractable syringe according to claim 11 wherein control of the rate of auto retraction of the needle is achieved by controlling the airflow in the plunger as the needle, or needle assembly, is retracted.

13. An auto-retractable syringe according to claim 12 wherein the method of claim 3 is utilized to control the rate of auto retraction of the needle or needle assembly.

14.-16. (canceled)
